SEQUENCE LISTING (1 sequence)

SEQ ID NO: 1 - DNA, 917 bp, Pezicula sp. SC1337
cgaggtcaccttgatagattgggggttgctggccagcatccgccgggcgtctgtagcgagagtatgtactacgcttagagccagacggcgccgccactgattttaaggcacaccgggaccggtgacgcccaagaccaagcagagcttgagggtgtaatgacgctcgaacaggcatgccccccggaataccaaggggcgcaatgtgcgttcaaagattcgatgattcactgaattctgcaattcacattacttatcgcatttcgctgcgttcttcatcgatgccagaaccaagagatccgttgttgaaagttttaactattatatagtactcagacgacactgacattcaggtttgggagtcctctggcgggcgcgccctagccggagccaggggcagggccggagccccgcggcccgccaaagcaacaaaggtataacgacacagggtgggaggtctacccgaagggcagagtctctgtaatgatccctccgcaggttcacctacggagcggtttacgttcttgcgaatccccagcacgtttccgcgccggcccgactatatcttaagcaggagaacctcctacccacaaccacttagtctgtgaacgttcccctggtctaggggcttcgctgcggatcgtccattccagcacctgactggatcctccggggttgttactctaccctgtgcagttaacacaggcccctactgcatccctacagtaggttagtacccagaggctttaggacttccccgcaattcgatcgtgttgcaactccgccgttgggcgtcgtcacttgcgtgcctccttatttagagtcgcgagagccccctttcggggtgcaaaggcttcctcaagttcgctgcagagagatcactgctctacggagaccttgtacttttttttattacttcc

(19) United States
(12) Patent Application Publication
(10) Pub. No.: US 2019/0166835 A1
(43) Pub. Date: Jun. 6, 2019

(54) Fungal Trisaccharide Ester Compounds and Use Thereof in Preparing Agents for Preventing and Controlling Plant Fungal Diseases

(71) Applicant: SOUTH CHINA BOTANICAL GARDEN, CHINESE ACADEMY OF SCIENCES, Guangzhou (CN)

(72) Inventors: Liangxiong XU, Guangzhou (CN); Xuewu DUAN, Guangzhou (CN); Xiaoyi WEI, Guangzhou (CN); Jinghua XUE, Guangzhou (CN); Linyan FENG, Guangzhou (CN); Ping WU, Guangzhou (CN)

(73) Assignee: SOUTH CHINA BOTANICAL GARDEN, CHINESE ACADEMY OF SCIENCES, Guangzhou (CN)

(21) Appl. No.: 16/322,947

(22) PCT Filed: Sep. 18, 2017
(86) PCT No.: PCT/CN2017/102047

§ 371 Date: Feb. 3, 2019

(30) Foreign Application Priority Data
Mar. 15, 2017 (CN) 201710154256.1

(51) Int. Cl. A01N 43/16; C07H 13/04; A23B 7/154

(52) U.S. Cl. CPC A01N 43/16; A23V 2002/00; A23B 7/154; C07H 13/04

(57) ABSTRACT

Fungal trisaccharide ester compounds and agents containing the fungal trisaccharide ester compounds as active ingredients for preventing and controlling plant fungal diseases. A Pezicula sp. strain SC1337 was isolated from endophytes of a branch of bald cypress. The strain SC1337 is capable of producing five compounds exhibiting significant inhibitory activities against twelve common plant pathogenic fungi and preservative effect on fruits. Thus, the strain SC1337 can be used to prepare the five compounds exhibiting significant inhibitory activities against twelve common plant pathogenic fungi and preservative effect on fruits and used for preventing and controlling plant pathogenic fungi and fruit preservation.

### CROSS REFERENCE TO RELATED APPLICATIONS

This application is the national phase entry of International Application No. PCT/CN2017/102047, filed on Sep. 18, 2017, which is based upon and claims priority to Chinese Patent Application No. 201710154256.1, filed on Mar. 15, 2017, the entire contents of which are incorporated herein by reference.

### TECHNICAL FIELD

The present invention relates to the field of natural products, and particularly to trisaccharide esters from fungal metabolites and use thereof in preparing agents for preventing and controlling plant fungal diseases.

### BACKGROUND

Plant diseases have always been one of the key constraints that limit the quality and yield of crops. Among plant diseases, 70% to 80% of the diseases are caused by infection with pathogenic fungi. On one hand, plant fungal diseases directly cause declines of crop yields, and rot and deterioration during storage and transportation of agricultural products, which results in great loss; on the other hand, some pathogenic fungi, when crops and agricultural products are infected, can secrete a variety of mycotoxins and harmful metabolites which are harmful to humans and animals, posing a great threat to the safety of agricultural products. At present, control of crop fungal diseases often relies on chemical control. Application of fungicides not only increases the cost, but also inevitably brings environmental pollution and chemical residue problems in agricultural products if they are applied repeatedly. The chemical control of major fungal diseases of the main crops in China mainly depends on a small number of fungicides, but there is a high risk that the pathogenic fungi will develop fungicide resistance and thereby the fungicide will lose their effectiveness. Therefore, it is of great significance to develop novel and efficient fungistats.

For traditional synthetic fungistats, as they have been used over a long period of time, the pathogenic fungi have developed partial resistance, resulting in the reduction in their effectiveness; also, since they are difficult to decompose, the residues will be spread to the surrounding environment, causing damage to the ecosystem and human health. In contrast, natural fungistats, as they exhibit relatively higher selectivity and are easy to decompose, such that there will be less residues and thereby they are relatively safe for human, livestock and beneficial organisms and bring less pollution, have been a hot research topic in the field of preventing and controlling plant diseases in recent years. The commonly used agricultural fungistats at present, such as validamycin, kasugamycin, polyoxins, Zhongshengmycin, Ningnanmycin and Shenqinmycin, are all derived from secondary metabolites of environmental microorganisms (mainly actinomycetes), but rarely from fungi. However, plant endophytic fungi produce a rich variety of secondary metabolites with diverse structures, which are also important resources for the development of novel and efficient microorganism-derived fungistats.

### SUMMARY

One object of the present invention is to provide five trisaccharide ester compounds which exhibit significant inhibitory activities against twelve common plant pathogenic fungi and preservative effect on fruits.

The trisaccharide ester compounds are compounds 1, 2, 3, 4 and 5, wherein the compound 1 is as shown in formula 1, the compound 2 is as shown in formula 2, the compound 3 is as shown in formula 3, the compound 4 is as shown in formula 4, and the compound 5 is as shown in formula 5.

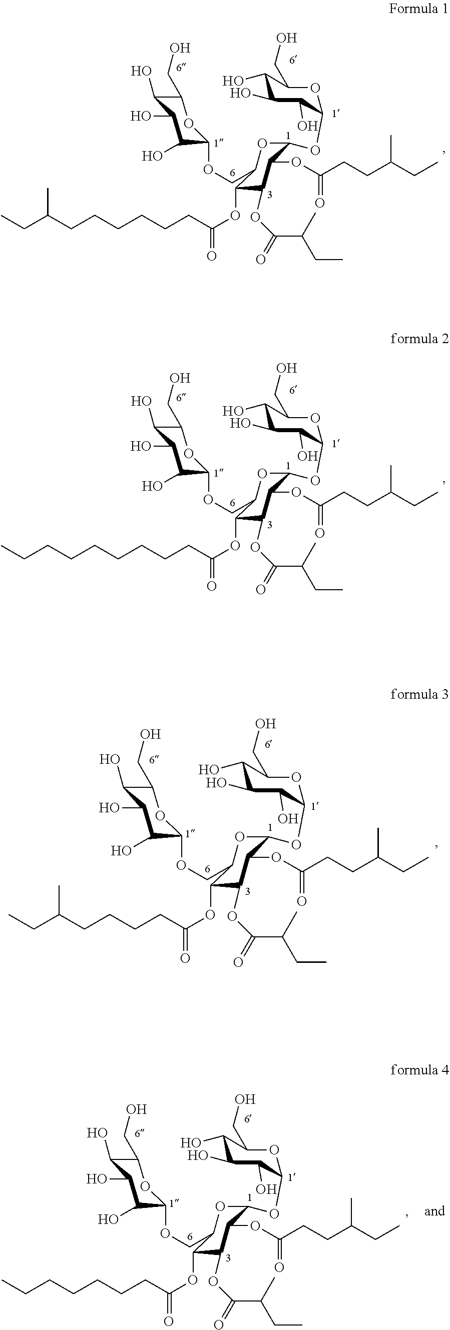

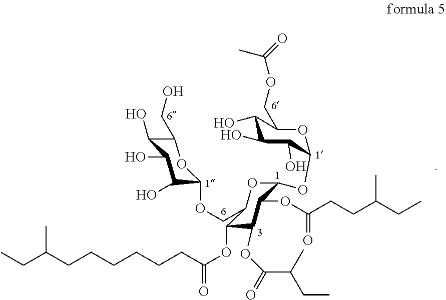

A second object of the present invention is to provide use of the above compounds 1, 2, 3, 4 and 5 in preparing agents for preventing and controlling fungal diseases.

The agents for preventing and controlling fungal diseases, are agents for preventing and controlling plant fungal diseases.

Also provided are agents for preventing and controlling fungal diseases, comprising the compound 1, 2, 3, 4 or 5, which is described in claim 1, as an active ingredient.

Preferably, the agents for preventing and controlling plant fungal diseases, are agents for preventing and controlling Alternaria sp., Botryospuaeria sp., Botrytis sp., Colletotrichum sp., Curvularia sp., Fusarium sp., Geotrichum sp., Helminthosporium sp., Penicillium sp., Peronophythora sp., Rhizoctonia sp., and Ustilaginoidea sp.

The agents for preventing and controlling plant fungal diseases, are agents for preventing and controlling early blight of tomatoes, ring rot of apples, grey mould of tomatoes, anthracnose of mangos, Curvularia lunata infection in bananas, fusarium wilt of bananas and watermelons, sour rot of citrus, southern corn leaf blight, blue mould of citrus, Peronophythora litchii infection in litchi, sheath blight of rice, or false smut of rice.

A third object of the present invention is to provide use of the compounds 1, 2, 3, 4 and 5 in preparing fruit preservatives.

Also provided are fruit preservatives, comprising the compound 1, 2, 3, 4 or 5, which is described in claim 1, as an active ingredient.

Preferably, the fruit preservatives are preservatives for citrus.

The inventors had isolated a Pezicula sp. strain SC1337 from endophytes of a branch of bald cypress. The strain SC1337 is capable of producing five compounds which exhibit significant inhibitory activities against twelve common plant pathogenic fungi and preservative effect on fruits. Thus, the strain can be used to prepare the five compounds which exhibit significant inhibitory activities against twelve common plant pathogenic fungi and preservative effect on fruits and used for preventing and controlling plant pathogenic fungi and fruit preservation, indicating a promising application potential.

The Pezicula sp. strain SC1337 has been deposited with the Guangdong Microbial Culture Collection Center (GDMCC), located at Guangdong Institute of Microbiology, No. 59 Building, No. 100 Xianliezhong Road, Guangzhou 510070, China on Feb. 15, 2017, and has been assigned the following accession number: GDMCC No. 60144.

### BRIEF DESCRIPTION OF THE DRAWINGS

FIG. 1 shows a colony of the Pezicula sp. strain SC1337.

FIG. 2 shows hyphae and spores of the Pezicula sp. strain SC1337.

### DETAILED DESCRIPTION OF THE EMBODIMENTS

The following embodiments are intended to further illustrate the invention and not to limit the invention.

### Embodiment 1

The Pezicula sp. strain SC1337 of the present invention was isolated from endophytes of a branch of bald cypress located at South China Botanical Garden.

Isolation method: The collected bald cypress branch sample was surface-sterilized with 75% ethanol for 30 seconds, and then sterilized with 10% (mass fraction) sodium hypochlorite solution for 3 minutes. The sample was washed with running sterile water for three times and then let dry. The sample was pressed onto a PDA medium which was then placed in a 28° C. incubator to allow incubation. After five days of incubation, the target colony was collected by scraping with a sterile toothpick. The PDA medium comprised, per litre, 6 g of potato extract, 20 g of glucose, 20 g of agar and the balance water, with a pH value of 6.0 to 6.5. Thereby, the Pezicula sp. strain SC1337 was obtained.

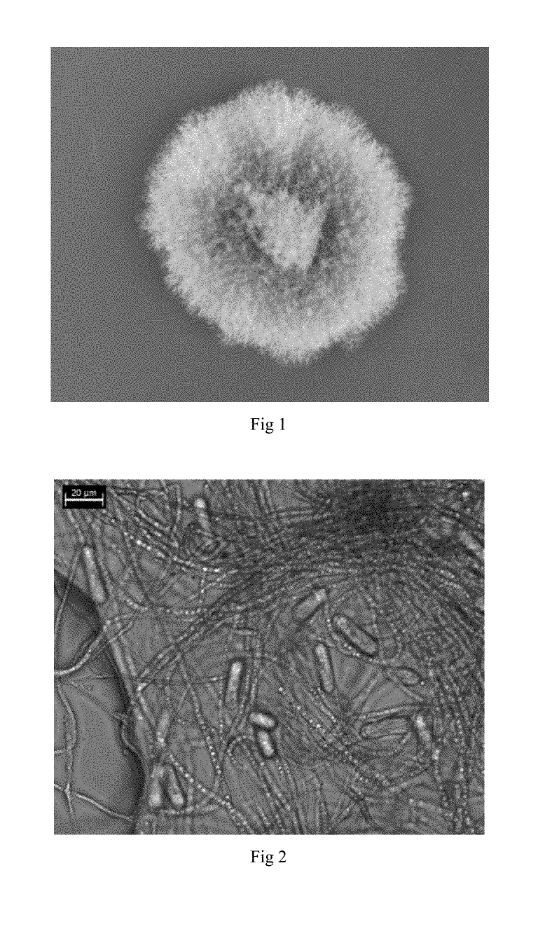

Taxonomic characteristics of the Pezicula sp. strain SC1337: The hyphae had a width of 2-3 μm. The spores were elliptical and have a size of (14-22)×(4-6) μm. Colonial morphology and hyphae morphology (together with spoors) are presented in FIG. 1 and FIG. 2 respectively. DNA was extracted from the mycelia by using the classical CTAB protocol. PCR amplification was conducted with ITS4 (5′-3′: TCC TCC GCT TAT TGA TAT GC) and ITS5 (5′-3′: GGA AGT AAA AGT CGT AAC AAG G) as primers, and within a system comprising 5 μL of 10×PCR buffer, 4 μL of Mg²⁺ (25 mM), 1 μL of dNTPs (10 mM), 2 μL of Primers (10 μM/each), 0.3 μL of Taq polymerase (5 U/μL), 5 μL of template DNA (10 ng/μL), and 32.7 μL of ddH₂O. PCR procedure: 94° C./5 min, 94° C./40 s, 57-52° C./1 min, 72° C./1 min, cycles 35, 72° C./6 min. The obtained DNA was sequenced and determined to be as SEQ ID NO: 1. The sequence was BLASTed against GenBank for homology search, and its internal transcribed spacer sequence (917 bp totally) showed a highest homology level to Pezicula cinnamomea, with a similarity of up to 96%. Accordingly, the fungus strain was named as Pezicula sp. strain SC1337. The Pezicula sp. strain SC1337 has been deposited with the Guangdong Microbial Culture Collection Center (GDMCC), located at Guangdong Institute of Microbiology, No. 59 Building, No. 100 Xianliezhong Road, Guangzhou 510075, China on Feb. 15, 2017, and has been assigned the following accession number: GDMCC No. 60144.

### Embodiment 2

Preparation of the five trisaccharide ester derivatives and their physicochemical and spectral data

The Pezicula sp. strain SC1337 was inoculated onto a wheat solid medium (a mixture of wheat and water in a mass ratio of 1:1.5) and stationary incubated in dark for 12 days so as to give a solid fermented culture.

The solid fermented culture (3.6 L) was then soaked in a same volume of 95% (v/v) ethanol aqueous solution three times, for 48 hours each time. All the solutions were combined, filtered and then subjected to vacuum concentration to remove the ethanol. Then the solution was added with water to a volume of 1 L in a volumetric flask, and extracted with a same volume of ethyl acetate three times, the extraction solutions were combined and concentrated to give 17.6 g of ethyl acetate extract.

The ethyl acetate extract was then loaded on a silica gel column (silica gel: 100 to 200 mesh, 300 g) gradiently eluted with chloroform/methanol with the volume ratio from 90:10 to 70:30. The fractions were detected by thin-layer chromatography (on a silica gel plate) and similar fractions were combined. Using chloroform/methanol (V/V=85/15) as the developing solvent, the fractions having a retention factor value of 0.3 to 0.4 on the plate were collected and subjected to vacuum concentration to give 1.69 g of fraction A, while the fractions having a retention factor value of 0.2 to 0.3 on the plate were collected and subjected to vacuum concentration to give 8.50 g of fraction B.

The fraction A was loaded onto a reversed phased silica gel column (Develosil ODS, 75 μm, 150 g, Fuji Chemical, Japan) eluted with a methanol aqueous solution having a volume fraction of 88%; the fractions which gave the major spot on thin-layer chromatography (silica gel plate) were collected, combined, and purified through high performance liquid chromatography (LC-6AD semi-preparative high performance liquid chromatography, with an RID-10A detector, manufactured by the Shimadzu Company, Japan; column: Shim-pack PRC-ODS, 10×250 mm, 4.5 μm), with a methanol aqueous solution having a volume fraction of 88% as the mobile phase and with a flow rate of 5 ml/min, to give a white powder compound 5 (0.030 g, t_{R}=60 min).

The fraction B was loaded onto the reversed phased silica gel column (Develosil ODS, 75 μm, 150 g) gradiently eluted with methanol aqueous solution with the volume ratio from 65% to 85%; the fraction eluted with 85% methanol was collected, concentrated and passed through high performance liquid chromatography (the device and column were identical to those previous described), with a methanol aqueous solution having a volume fraction of 87% as the mobile phase and with a flow rate of 5 ml/min, to give four white powder products: compound 1 (0.122 g, t_{R}=55 min), compound 2 (0.120 g, t_{R}=45 min), compound 3 (0.010 g, t_{R}=33 min) and compound 4 (0.122 g, t_{R}=28 min).

Compound 1 was in the form of white powder, having a molecular formula as C₄₁H₇₂O₁₉. Positive HRESIMS data: m/z 891.4552 [M+Na]⁺ (calcd for C₄₁H₇₂O₁₉ Na, 891.4560). Positive ESIMS data: m/z 891 [M+Na]⁺, 907 [M+K]⁺. Negative ESIMS data: m/z 867 [M−H]⁻, 903 [M+Cl]⁻. See table 1 for ¹H NMR data and ¹³C NMR data (solvent: deuterated methanol). Through the above spectroscopic data and the 2D-NMR test, the compound 1 was determined to be 6-O-β-L-mannosyl-3-O-(2-methylbutyryl)-4-O-(8-methyl decanoyl)-2-O-(4-methylhexanoyl)trehalose, as shown in formula 1.

Compound 2 was in the form of white powder, having a molecular formula as C₄₀H₇₀O₁₉. Positive HRESIMS data: m/z 877.4411 [M+Na]⁺ (calcd for C₄₀H₇₀O₁₉Na, 877.4404). Positive ESIMS data: m/z 877 [M+Na]⁺. Negative ESIMS data: m/z 853 [M−H]⁻, 889 [M+Cl]⁻. See table 1 for ¹H NMR data and ¹³C NMR data (solvent: deuterated methanol). Through the above spectroscopic data and the 2D-NMR test, the compound 2 was determined to be 4-O-decanoyl-6-O-β-L-mannosyl-3-O-(2-methylbutyryl)-2-O-(4-methylhexanoyl)trehalose, as shown in formula 2.

Compound 3 was in the form of white powder, having a molecular formula as C₃₉H₆₈O₁₉. Positive HRESIMS data: m/z 863.4255 [M+Na]⁺ (calcd for C₃₉H₆₈O₁₉Na, 863.4247). Positive ESIMS data: m/z 863 [M+Na]⁺. Negative ESIMS data: m/z 839 [M−H]⁻, 875 [M+Cl]⁻. See table 1 for ¹H NMR data and ¹³C NMR data (solvent: deuterated methanol). Through the above spectroscopic data and the 2D-NMR test, the compound 3 was determined to be 6-O-β-L-mannosyl-3-O-(2-methylbutyryl)-2-O-(4-methylhexanoyl)-4-O-(6-methyloctanoyl)trehalose, as shown in formula 3.

Compound 4 was in the form of white powder, having a molecular formula as C₃₈H₆₆O₁₉. Positive HRESIMS data: m/z 849.4088 [M+Na]⁺ (calcd for C₃₈H₆₆O₁₉Na, 849.4091). Positive ESIMS data: m/z 849 [M+Na]⁺. Negative ESIMS data: m/z 825 [M−H]. See table 2 for ¹H NMR data and ¹³C NMR data (solvent: deuterated methanol). Through the above spectroscopic data and the 2D-NMR test, the compound 4 was determined to be 6-O-β-L-mannosyl methylbutyryl)-2-O-(4-methylhexanoyl)-4-O-octanoyltrehalose, as shown in formula 4.

Compound 5 was in the form of white powder, having a molecular formula as C₄₃H₇₄O₂₀. Positive ESIMS data: m/z 933 [M+Na]⁺, 949 [M+K]⁺. Negative ESIMS data: m/z 909 [M−H]⁻, 945 [M+Cl]⁻. See table 2 for ¹H NMR data and ¹³C NMR data (solvent: deuterated methanol). Through the above spectroscopic data and the 2D-NMR test, the compound 5 was determined to be 6-O-β-L-mannosyl-3-O-(2-methylbutyryl)-4-O-(8-methyldecanoyl)-2-O-(4-methylhexanoyl)-6′-O-acetyltrehalose, as shown in formula 5.

TABLE 1
¹H NMR data and ¹³C NMR data for compounds 1 to 3

| | Compound 1 | | Compound 2 | | Compound 3 | |
|---|---|---|---|---|---|---|
| position | ¹H (J in Hz) | ¹³C | ¹H (J in Hz) | ¹³C | ¹H (J in Hz) | ¹³C |

trehalose

| | | | | | | |
|---|---|---|---|---|---|---|
| 1 | 5.36 (d, 3.7) | 91.56 | 5.36 (d, 3.7) | 91.56 | 5.36 (d, 3.7) | 91.57 |
| 2 | 5.03 (dd, 10.2, | 70.29 | 5.03 (dd, 10.2, 3.7) | 70.28 | 5.03 (dd, 10.2, 3.7) | 70.27 |
| | 3.7) | | | | | |
| 3 | 5.63 (dd, 10.0, | 70.00 | 5.63 (dd, 10.1, 9.6) | 70.00 | 5.63 (dd, 10.1, 9.4) | 70.00 |
| | 9.5) | | | | | |
| 4 | 5.21 (dd, 10.0, | 68.56 | 5.20 (dd, 10.1, 9.6) | 68.55 | 5.21 (dd, 10.1, 9.4) | 68.54 |
| | 9.5) | | | | | |
| 5 | 4.45 (dd, 10.0, | 68.94 | 4.45 (ddd, 10.1, | 68.93 | 4.45 (ddd, 10.1, | 68.92 |
| | 4.8, 2.0) | | 4.6, 2.0) | | 4.8, 2.0) | |
| 6 | 4.00 (dd, 11.6, | 67.54 | 4.00 (dd, 11.6, 2.0); | 67.53 | 4.01 (dd, 11.6, 2.0); | 67.51 |
| | 2.0); 3.62 (dd, | | 3.61 (dd, 11.6, 4.6) | | 3.61 (dd, 11.6, 4.8) | |
| | 11.6, 5.0) | | | | | |
| 1′ | 4.45 (d, 0.6) | 101.17 | 4.45 (br s) | 101.18 | 4.45 (d, 0.7) | 101.18 |
| 2′ | 3.93 (d, 3.2) | 70.78 | 3.93 (d, 3.2) | 70.78 | 3.93 (d, 3.3) | 70.79 |
| 3′ | 3.43 (dd, 9.4, 3.2) | 73.79 | 3.43 (dd, 9.4, 3.2) | 73.79 | 3.43 (dd, 9.4, 3.2) | 73.79 |
| 4′ | 3.58 (t, 9.5) | 67.04 | 3.58 (t, 9.5) | 67.04 | 3.58 (t, 9.5) | 67.05 |
| 5′ | 3.19 (ddd, 9.6, | 76.95 | 3.19 (m) | 76.96 | 3.19 (ddd, 9.6, 5.9, | 76.96 |
| | 5.8, 2.4) | | | | 2.4) | |
| 6′ | 3.88 (dd, 11.8, | 61.42 | 3.88 (dd, 11.8, 2.4); | 61.41 | 3.88 (dd, 11.8, 2.4); | 61.42 |
| | 2.4); 3.73 (m) | | 3.73 (dd, 11.8, 6.0) | | 3.74 (dd, 11.8, 5.9) | |

6-O-β-L-mannosyl group

| | | | | | | |
|---|---|---|---|---|---|---|
| 1″ | 5.11 (d, 3.7) | 95.04 | 5.11 (d, 3.8) | 95.04 | 5.10 (d, 3.7) | 95.05 |
| 2″ | 3.53 (dd, 9.8, 3.8) | 71.53 | 3.53 (dd, 9.8, 3.8) | 71.53 | 3.53 (dd, 9.8, 3.7) | 71.53 |
| 3″ | 3.80 (dd, 9.8, 9.2) | 73.08 | 3.79 (dd, 9.8, 9.0) | 73.08 | 3.80 (dd, 9.8, 9.2) | 73.08 |
| 4″ | 3.36 (t, 9.2) | 70.16 | 3.36 (t, 9.0) | 70.16 | 3.36 (t, 9.2) | 70.16 |
| 5″ | 3.68 (dd, 8.0, 2.1) | 73.13 | 3.68 (m) | 73.14 | 3.68 (m) | 73.14 |
| 6″ | 3.74 (m); | 61.08 | 3.73 (m); 3.68 (m) | 61.08 | 3.74 (m); 3.68 (m) | 61.09 |
| | 3.68 (m) | | | | | |

2-O-(4-methylhexanoyl) group

| | | | | | | |
|---|---|---|---|---|---|---|
| −1 | | 172.98 | | 172.99 | | 172.98 |
| −2 | 2.43 (ddd, 16.1, | 31.17 | 2.43 (ddd, 16.1, | 31.17 | 2.43 (ddd, 16.2, | 31.17 |
| | 10.3, 5.3); | | 10.3, 5.3); 2.30 (m) | | 10.3, 5.3); 2.31 (m) | |
| | 2.30 (m) | | | | | |
| −3 | 1.63 (m); | 30.87 | 1.63 (m); 1.41 (m) | 30.87 | 1.63 (m); 1.41 (m) | 30.87 |
| | 1.41 (m) | | | | | |
| −4 | 1.34 (m) | 33.83 | 1.34 (m) | 33.83 | 1.35 (m) | 33.83 |
| −5 | 1.33 (m) | 28.82 | 1.33 (m) | 28.81 | 1.37 (m); 1.18 (m) | 28.81 |
| −6 | 0.90 (t, 7.5) | 10.37 | 0.90 (t, 7.5) | 10.23 | 0.90 (t, 7.5) | 10.22 |
| −7 | 0.88 (d, 7.0) | 17.78 | 0.88 (d, 7.0) | 17.77 | 0.89 (d, 6.5) | 17.77 |

3-O-(2-methylbutyryl) group

| −1 | | 175.66 | | 175.67 | | 175.67 |
|---|---|---|---|---|---|---|
| −2 | 2.33 (m) | 40.95 | 2.32 (m) | 40.96 | 2.32 (m) | 40.97 |
| −3 | 1.63 (m); | 26.22 | 1.63 (m); 1.44 (m) | 26.21 | 1.63 (m); 1.44 (m) | 26.21 |
| | 1.44 (m) | | | | | |
| −4 | 0.90 (t, 7.5) | 10.70 | 0.89 (t, 7.5) | 10.69 | 0.90 (t, 7.5) | 10.69 |
| −5 | 1.09 (d, 7.0) | 15.66 | 1.09 (d, 7.0) | 15.65 | 1.09 (d, 7.0) | 15.66 |
| | 4-O-(8-methyldecanoyl) | | 4-O-decanoyl | | 4-O-(6-methyloctanoyl) | |
| | group | | group | | group | |
| −1 | | 172.65 | | 172.65 | | 172.64 |
| −2 | 2.35 (m); | 33.53 | 2.35 (m); 2.28 (m) | 33.52 | 2.37 (m); 2.30 (m) | 33.54 |
| | 2.29 (m) | | | | | |
| −3 | 1.58 (m) | 24.44 | 1.58 (m) | 24.43 | 1.57 (m) | 24.73 |
| −4 | 1.48-1.28 (m) | *29.36 | 1.50-1.26 (m) | *29.15 | 1.35 (m) | 26.38 |
| −5 | 1.48-1.28 (m) | *29.19 | 1.50-1.26 (m) | *29.01 | 1.35 (m); 1.14 (m) | 36.00 |
| −6 | 1.33 (m) | 26.62 | 1.50-1.26 (m) | *29.01 | 1.35 (m) | 34.10 |
| −7 | 1.33 (m); | 36.35 | 1.50-1.26 (m) | *28.87 | 1.34 (m); 1.18 (m) | 29.15 |
| | 1.13 (m) | | | | | |
| −8 | 1.34 (m) | 34.29 | 1.31 (m) | 31.64 | 0.90 (t, 7.2) | 10.33 |
| −9 | 1.33 (m); 1.18 | 28.91 | 1.33 (m) | 22.33 | 0.89 (d, 6.5) | 18.16 |
| −10 | 0.90 (t, 7.3) | 10.23 | 0.92 (t, 7.2) | 13.04 | | |
| −11 | 0.88 (d, 7.0) | 18.24 | | | | |

*The signals of the corresponding positions are interchangeable

TABLE 2
¹H NMR data and ¹³C NMR data for compounds 4 and 5

| | Compound 4 | | Compound 5 | |
|---|---|---|---|---|
| position | ¹H (J in Hz) | ¹³C | ¹H (J in Hz) | ¹³C |

trehalose

| | | | | |
|---|---|---|---|---|
| 1 | 5.36 (d, 3.8) | 91.57 | 5.36 (d, 3.7) | 91.61 |
| 2 | 5.03 (dd, 10.2, 3.8) | 70.29 | 5.03 (dd, 10.2, 3.7) | 70.26 |
| 3 | 5.63 (dd, 10.1, 9.4) | 70.00 | 5.63 (dd, 10.2, 9.7) | 69.94 |
| 4 | 5.21 (dd, 10.1, 9.4) | 68.55 | 5.20 (dd, 10.2, 9.7) | 68.53 |
| 5 | 4.45 (ddd, 10.1, 4.6, 2.0) | 68.93 | 4.43 (m) | 68.87 |
| 6 | 4.01 (dd, 11.6, 2.0); 3.61 (dd, 11.6, 4.6) | 67.53 | 3.97 (dd, 11.5, 1.8); 3.58 (m) | 67.55 |
| 1′ | 4.45 (d, 0.5) | 101.19 | 4.45 (br s) | 101.14 |
| 2′ | 3.93 (d, 3.2) | 70.79 | 3.94 (d, 3.2) | 70.56 |
| 3′ | 3.43 (dd, 9.4, 3.2) | 73.80 | 3.45 (dd, 9.3, 3.2) | 73.59 |
| 4′ | 3.58 (t, 9.5) | 67.06 | 3.58 (t, 9.3) | 67.15 |
| 5′ | 3.19 (ddd, 9.6, 5.9, 2.4) | 76.97 | 3.37 (m) | 74.42 |
| 6′ | 3.88 (dd, 11.8, 2.4); 3.73 (m) | 61.43 | 4.41 (dd, 11.8, 1.9); 4.26 (dd, 11.8, 6.4) | 63.63 |

6-O-β-L-mannosyl group

| | | | | |
|---|---|---|---|---|
| 1″ | 5.10 (d, 3.8) | 95.05 | 5.10 (d, 3.7) | 95.12 |
| 2″ | 3.53 (dd, 9.8, 3.8) | 71.54 | 3.53 (dd, 9.8, 3.7) | 71.55 |
| 3″ | 3.80 (dd, 9.8, 9.0) | 73.09 | 3.80 (dd, 9.8, 9.2) | 73.10 |
| 4″ | 3.36 (t, 9.0) | 70.17 | 3.37 (m) | 70.16 |
| 5″ | 3.68 (m) | 73.14 | 3.68 (m) | 73.14 |
| 6″ | 3.73 (m); 3.68 (m) | 61.09 | 3.74 (m); 3.68 (m) | 61.10 |

2-O-(4-methylhexanoyl) group

| | | | | |
|---|---|---|---|---|
| -1 | | 172.98 | | 172.98 |
| -2 | 2.43 (ddd, 16.0, 10.0, 4.8); 2.29 (m) | 31.18 | 2.43 (ddd, 16.0, 10.3, 5.3); 2.30 (m) | 31.18 |
| -3 | 1.63 (m); 1.41 (m) | 30.88 | 1.63 (m); 1.41 (m) | 30.87 |
| -4 | 1.35 (m) | 33.84 | 1.34 (m) | 33.83 |
| -5 | 1.37 (m); 1.18 (m) | 28.82 | 1.33 (m) | 28.81 |
| -6 | 0.94 (t, 7.5) | 10.23 | 0.90 (t, 7.5) | 10.36 |
| -7 | 0.88 (d, 6.4) | 17.78 | 0.88 (d, 7.0) | 17.78 |

3-O-(2-methylbutyryl) group

| F-1 | | 175.67 | | 175.63 |
|---|---|---|---|---|
| -2 | 2.32 (m) | 40.97 | 2.32 (m) | 40.96 |
| -3 | 1.63 (m); 1.43 (m) | 26.23 | 1.63 (m); 1.44 (m) | 26.21 |
| -4 | 0.89 (t, 7.5) | 10.69 | 0.90 (t, 7.5) | 10.70 |
| -5 | 1.09 (d, 7.0) | 15.64 | 1.09 (d, 7.0) | 15.67 |
| | 4-O-octanoyl group | | 4-O-(8-methyldecanoyl) group | |
| -1 | | 172.64 | | 172.60 |
| -2 | 2.34 (m); 2.29 (m) | 33.53 | 2.35 (m); 2.29 (m) | 33.55 |
| -3 | 1.58 (m) | 24.44 | 1.58 (m) | 24.45 |
| -4 | 1.36-1.30 (m) | *28.69 | 1.48-1.28 (m) | *29.34 |
| -5 | 1.36-1.30 (m) | *28.84 | 1.48-1.28 (m) | *29.19 |
| -6 | 1.32 (m) | 31.44 | 1.33 (m) | 26.62 |
| -7 | 1.34 (m) | 22.29 | 1.33 (m); 1.12 (m) | 36.34 |
| -8 | 0.94 (t, 7.5) | 13.01 | 1.34 (m) | 34.29 |
| -9 | | | 1.33 (m); 1.17 (m) | 28.91 |
| -10 | | | 0.90 (t, 7.3) | 10.23 |
| -11 | | | 0.89 (d, 7.0) | 18.24 |

6′-acetyl group

| | | |
|---|---|---|
| -1 | | 171.46 |
| -2 | 2.09 (s) | 19.44 |

*The signals of the corresponding positions are interchangeable

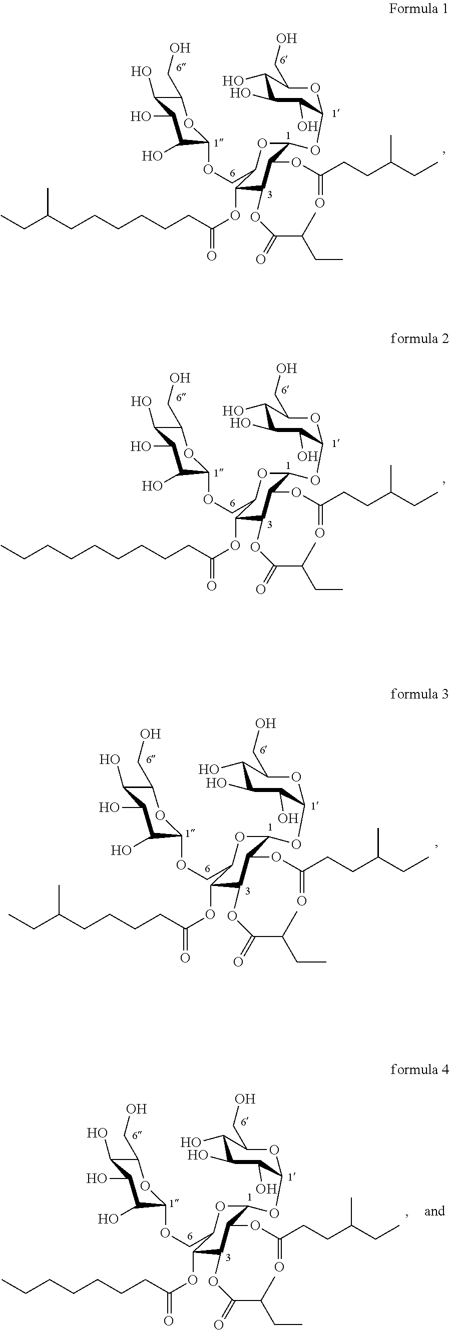

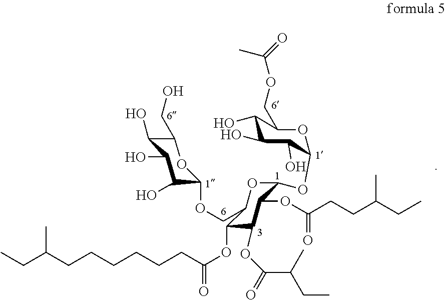

### Embodiment 3

Experiment of in vitro inhibitory activities of the trisaccharide ester compounds 1 to 5 obtained in embodiment 2 against common plant pathogenic fungi of twelve genus.

The fungi used in the experiments include: 1) Alternaria solani (causing early blight of tomatoes), 2) Botryospuaeria berengeriana (causing ring rot of apples), 3) Botrytis cinerea (causing grey mould of tomatoes), 4) Colletotrichum gloeosporioides (causing anthracnose of mangos), 5) Curvularia lunata (which will infect bananas), 6) Fusarium oxysporium (causing fusarium wilt of bananas and watermelons), 7) Geotrichum citri-aurantii (causing sour rot of citrus), 8) Helminthosporium maydis (causing southern corn leaf blight), 9) Penicillium italicum (causing blue mould of citrus), 10) Peronophythora litchii (which will infect litchi), 11) Rhizoctonia solani (causing sheath blight of rice), and 12) Ustilaginoidea virens (causing false smut of rice).

The antifungal activities of the compounds 1 to 5 were determined by the agar diffusion test. The fungi were activated and respectively seeded onto petri dishes containing PDA solid culture medium, and spread uniformly with T-shaped spreaders. Each compound was dissolved in methanol to give a 50 mg/mL solution. 10 μL of the solution was then added dropwise onto a filter paper (with a diameter of 6 mm) and let dry in the clean bench such that each filter paper contained 500 μg of the compound eventually. The filter papers were then pressed to the pertri dishes mentioned above and left to incubate in dark at 28° C. for 72 or 96 hours, and then the inhibition widths were measured (a inhibition width refers to an average diameter of the zones of inhibition), wherein filter papers containing a same volume of methanol were used as negative controls.

Results showed that a trisaccharide ester extract and the compound 1 to 5 were all capable of inhibiting the growth of all the fungi mentioned above, with inhibition widths from 10 mm to 22 mm, indicating broad-spectrum inhibitory activities against fungi. See table 3 for the results. The trisaccharide ester extract was a mixture containing the compounds 1 to 4, with a mass ratio of about 60:35:4:1.

TABLE 3
In vitro inhibitory activities of the trisaccharide ester extract and
the compounds 1 to 5 against twelve plant pathogenic fungi

| | | Zone of inhibition (mm) | | | | | |
|---|---|---|---|---|---|---|---|
| | Fungi | Extract | 1 | 2 | 3 | 4 | 5 |
| 1 | Alternaria solani | 12 | 12 | 14 | 13 | 12 | 10 |
| 2 | Botryospuaeria berengeriana | 20 | 16 | 14 | 12 | 10 | 8 |
| 3 | Botrytis cinerea | 18 | 15 | 14 | 16 | 10 | 9 |
| 4 | Colletotrichum gloeosporioides | 12 | 12 | 10 | 13 | 14 | 8 |
| 5 | Curvularia lunata | 14 | 12 | 15 | 12 | 15 | 16 |
| 6 | Fusarium oxysporium | 10 | 10 | 12 | 13 | 10 | 8 |
| 7 | Geotrichum citri-aurantii | 16 | 17 | 15 | 12 | 12 | 15 |
| 8 | Helminthosporium maydis | 20 | 22 | 18 | 19 | 18 | 18 |
| 9 | Penicillium italicum | 22 | 20 | 22 | 17 | 18 | 14 |
| 10 | Peronophythora litchii | 15 | 13 | 12 | 10 | 12 | 10 |
| 11 | Rhizoctonia solani | 15 | 18 | 16 | 15 | 18 | 12 |
| 12 | Ustilaginoidea virens | 20 | 22 | 20 | 18 | 20 | 16 |

### Embodiment 4

Experiment of the trisaccharide ester extract obtained in embodiment 2 in the preservation of citrus in postharvest storage

Samples for the experiment: the trisaccharide ester extract (containing the compounds 1 to 4, with a mass ratio of about 60:35:4:1) obtained in embodiment 2 was dissolved in a small volume of ethanol and diluted with water to give treating solutions (200, 400 and 600 mg/L).

The citrus fruits were soaked in the treating solutions for 5 minutes and left to dry. The fruits were then put in plastic baskets (the plastic baskets were surface-sterilized with 75% ethanol and left to dry in advance), sealed in polyethylene bags with a thickness of 0.02 mm, and stored at 25° C. for 15 days. The fruits were observed and their infection indexes were recorded. Calculation of the infection index: infection index=Σ(infection level×amount of fruits of the level)/(5×total amount of fruits)×100%; infection level: 0 refers to “no obvious infection”, 1 refers to “slight infection”, 2 refers to “infection area smaller than or equal to ¼ of fruit surface”; 3 refers to “infection area larger than ¼ and smaller than ½ of fruit surface”, and 4 refers to “infection area larger than or equal to ½ of fruit surface”. Three replicates were set for each treating solution, and 60 fruits for each replicate.

Fruits for the experiments: citrus reticulata blanco cv.shiyueju, harvested from Conghua Orchard.

See table 4 for the results. The trisaccharide ester extract had exhibited significant inhibitory effect on the fungal growth on the citrus in postharvest storage, higher than that of the common chemical fungicide Thiram and similar to that of Tecto, indicating a significant preservation effect. The preservation effect of the trisaccharide ester extract was slightly lower than that of the chemical fungicide Bellkute, but Bellkute had already been banned in European Union on 2004 for its high toxicity.

TABLE 4
Inhibitory activity of the trisaccharide ester extract against
pathogenic fungi in the postharvest storage of citrus fruits

| | Infection index (%) | | | | |
|---|---|---|---|---|---|
| | Concen- | | | | |
| Blank | tration | | | | |
| control | (mg/L) | Extract | Tecto | Bellkute | Thiram |
| 47.5 ± 3.38 | 200 | 16.0 ± 1.9 | 15.7 ± 1.2 | 3.66 ± 0.5 | 29.0 ± 1.9 |
| | 400 | 5.65 ± 1.0 | 12.5 ± 1.8 | 3.07 ± 0.5 | 31.4 ± 0.9 |
| | 600 | 6.5 ± 2.1 | 3.63 ± 0.4 | 3.40 ± 0.7 | 29.2 ± 1.0 |

What is claimed is:

1. (canceled)

2. A method of preparing an agent for preventing and controlling plant fungal diseases, comprising: using a trisaccharide ester compound as an active ingredient to prepare the agent wherein the trisaccharide ester compound is represented by a formula selected from the group consisting of

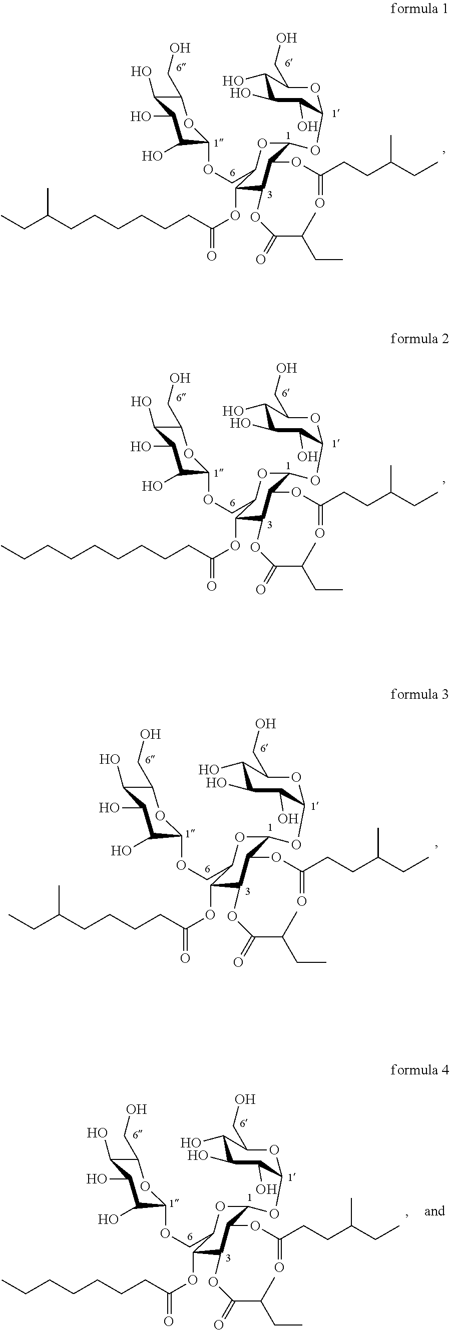

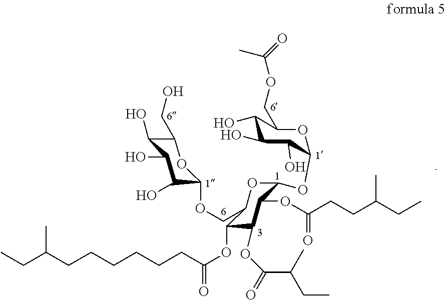

3. (canceled)

4. The method according to claim 2, wherein the agent is for preventing and controlling Alternaria sp., Botryospuaeria sp., Botrytis sp., Colletotrichum sp., Curvularia sp., Fusarium sp., Geotrichum sp., Helminthosporium sp., Penicillium sp., Peronophythora sp., Rhizoctonia sp., or Ustilaginoidea sp.

5. An agent for preventing and controlling plant fungal diseases, comprising a trisaccharide ester compound as an active ingredient; wherein the trisaccharide ester compound is represented by a formula selected from the group consisting of

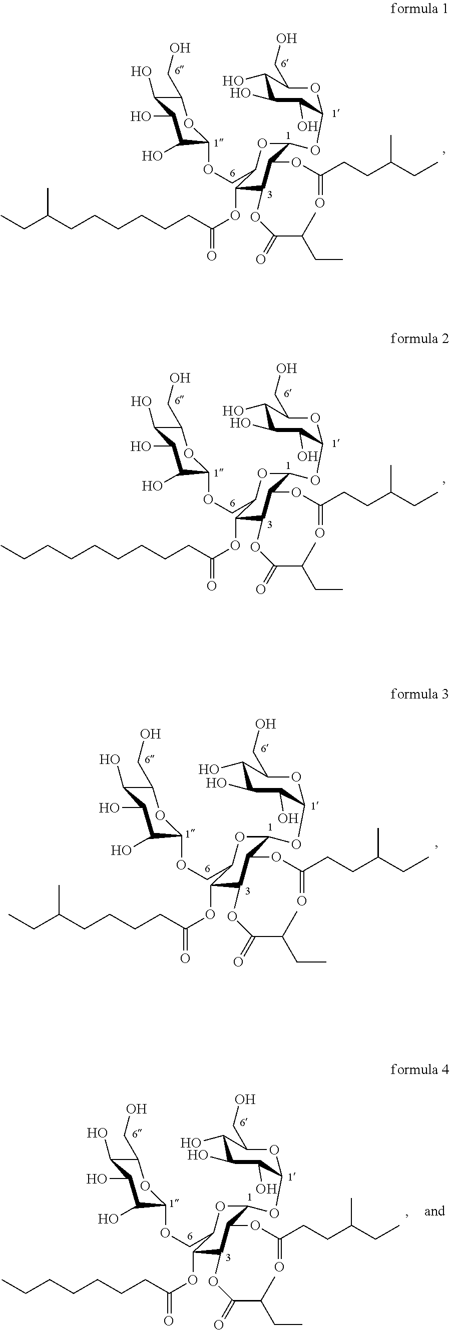

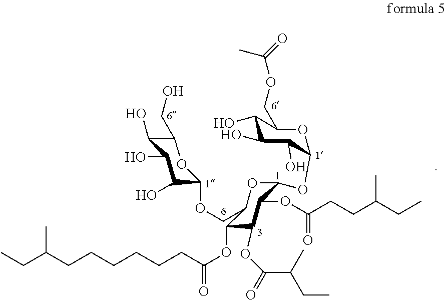

6. The agent for preventing and controlling the plant fungal diseases according to claim 5, wherein the agent is for preventing and controlling Alternaria sp., Botryospuaeria sp., Botrytis sp., Colletotrichum sp., Curvularia sp., Fusarium sp., Geotrichum sp., Helminthosporium sp., Penicillium sp., Peronophythora sp., Rhizoctonia sp., or Ustilaginoidea sp.

7. The agent for preventing and controlling the plant fungal diseases according to claim 5, wherein the plant fungal diseases comprise early blight of tomatoes, ring rot of apples, grey mould of tomatoes, anthracnose of mangos, Curvularia lunata infection in bananas, fusarium wilt of bananas and watermelons, sour rot of citrus, southern corn leaf blight, blue mould of citrus, Peronophythora litchii infection in litchi, sheath blight of rice, and false smut of rice.

8. (canceled)

9. (canceled)
